(19) United States
(12) Patent Application Publication
(10) Pub. No.: US 2011/0163300 A1
(43) Pub. Date: Jul. 7, 2011

(54) ORGANIC LIGHT-EMITTING MATERIAL, ORGANIC LIGHT-EMITTING ELEMENT USING THE SAME AND METHOD OF FORMING THE SAME

(75) Inventors: Hsin-Fei Meng, Hsinchu (TW); Sheng-Fu Horng, Hsinchu (TW); Hsin-Rong Tseng, Hsinchu (TW); Chia-Da Yu, Hsinchu (TW); Chung-Lin Yeh, Hsinchu (TW); Sheng-Yang Huang, Hsinchu (TW); Feng-Wen Yen, Hsinchu (TW); I-Feng Lin, Hsinchu (TW)

(73) Assignees: NATIONAL CHIAO TUNG UNIVERSITY, Hsinchu (TW); LUMINESCENCE TECHNOLOGY CORP., Hsin-Chu (TW)

(21) Appl. No.: 12/829,964

(22) Filed: Jul. 2, 2010

(30) Foreign Application Priority Data
Jan. 6, 2010 (TW) 99100150

(51) Int. Cl. H01L 51/54; H01L 51/52; H01L 51/56; C07C 13/66; C07C 211/54

(52) U.S. Cl. 257/40; 438/46; 585/27; 564/428; 257/E51.018; 257/E51.026

(57) ABSTRACT

The present invention provides compound of formula (I)

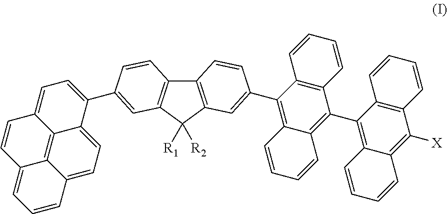

wherein each substituent is defined in the specification. The compound may be used, in combination with other organic light-emitting materials, in a light-emitting layer of an organic light-emitting element. The present invention also provides an organic light-emitting element including a first electrode, a second electrode and at least three layers of organic material layers disposed between the first electrode and the second electrode, wherein the layer used as a light-emitting layer contains a compound of formula (I). Further, an all-solution process, which is used for fabricating the organic light-emitting element of the present invention, has the advantages such as avoiding miscibility among the layers to fabricate an element with a large surface area and lower production cost.

### FIELD OF INVENTION

The present invention relates to organic light-emitting materials, and more particularly, to an organic light-emitting material for a light-emitting layer of an organic light-emitting element, an organic light-emitting element using the same and a method of forming the same.

### BACKGROUND OF THE INVENTION

In the developments of organic conductors, insulators and semiconductor materials, organic semiconductor materials, such as organic light-emitting devices (OLED), organic light emitting diodes (LED), solar cells, organic transistors and organic photodetectors, are important for the electronic and photoelectronic elements. Generally, OLEDs are classified into small molecular OLEDs and macromolecular OLEDs. A small molecular dye or pigment is a host material in a small molecular OLED, whereas a conjugative macromolecule is a host material in a macromolecular OLED. Currently, a vapor deposition process is performed on typical small molecular light-emitting diodes to prepare multi-layered structures. However, in the process, a highly vacuum chamber is required to perform thermal vapor deposition, and material usage efficiency is low. Thus, the cost of the vapor deposition process is very high. Further, the vapor deposition process has slow processing rate due to the complexity of the operation, and is not suitable for fabricating an element or device having a large surface area. As such, the small molecular OLEDs are mainly used in small-sized panels at the current stage. The conjugative macromolecule is typically obtained by forming a solution with an organic solvent, and then performing liquid molding. As compared with the small molecular OLEDs, the macromolecular OLEDs are formed by a solution process so as to lower product cost and maximize the surface areas. Nevertheless, due to the miscibility among layers as caused by the solution process, the macromolecular OLEDs are generally mono-layered, such that the products cannot meet the industrial demands.

Since the synthesis and purification of the material of a macromolecular OLED is not readily applicable to small molecules, small molecular materials are used in the solution process to prepare a multi-layered light-emitting diode so as to reduce the product cost and maximize the surface area thereof. Some improved methods are reported to achieve a multi-layered structure and to solve a problem related to the miscibility observed in a solution process. For example, US Patent Application Publication No. 20060029725 discloses that a first organic layer is insoluble in a solution used to deposit a second organic layer. However, such prior art does not have general applicability since it uses cross-linked molecules as the first organic layer to avoid dissolution, so as to overcome the miscibility among layers. Further, the publication on Applied Physics Letters, 92, 263301 (2008) only discloses a monolayer of small molecules, without mentioning a multi-layered structure to increase the efficiency of the OLED. The publication on Applied Physics Letters, 92, 063302 (2008) discloses adding small molecules for an electron transport layer and a light-emitting layer, but the efficiency and performance of the OLED are poor. Moreover, the publication on Applied Physics Letters, 92, 093307 (2008) discloses using an adhesive method, which does not provide a good control of the thickness and filming characteristics of each layer.

Although the above methods have been developed for improving a solution process, there still exist many drawbacks. Therefore, an urgent issue to be resolved in the industry is how to apply small molecular light-emitting materials to a solution process and fabricating an organic light-emitting element having a multi-layered film structure.

### SUMMARY OF THE INVENTION

The present invention provides a compound of formula (I):

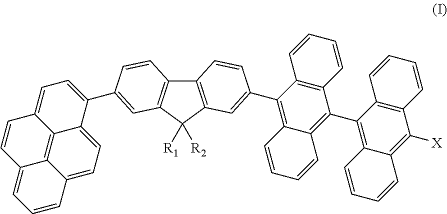

wherein R₁ and R₂ are each a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group. The compound of formula (I) of the present invention can be used as a light-emitting layer of an organic light-emitting element. Specifically, the compound is used as a host material for the light-emitting layer. The present invention further provides a compound of formula (II):

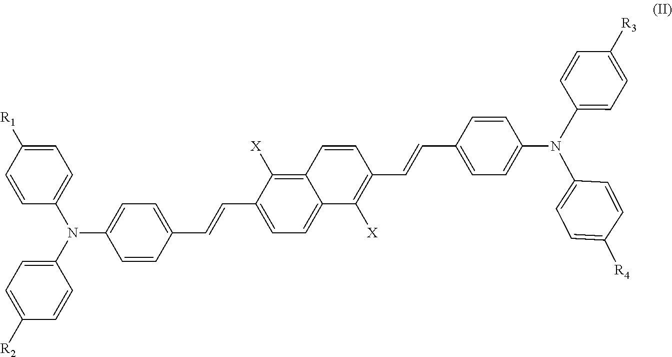

wherein R₁, R₂, R₃, and R₄ each have a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group.

The present invention provides an organic light-emitting element, comprising: a first electrode; a second electrode, a light-emitting layer disposed between the first organic electrode and the second electrode; a first carrier transport layer formed between the light-emitting layer and the first electrode; and a second carrier transport layer formed between the light-emitting layer and the second electrode, wherein the light-emitting layer comprises a compound of formula (I) and a compound of formula (II).

The present invention further provides a method for fabricating an organic light-emitting element, comprising the steps of: providing a substrate having a first electrode formed on a surface thereof and a first carrier transport layer formed on the first electrode; providing a solution of organic molecules on the first carrier transport layer; coating the solution of organic molecules on the substrate with a scraper to form a wet coating layer; heating the wet coating layer to remove the solvent to form a light-emitting layer; forming a second carrier transport layer on the light-emitting layer; and forming a second electrode on the second carrier transport layer, wherein the solution of organic molecules contains a compound of formula (I) and a compound of formula (II) of the present invention.

The small molecular compounds of the present invention are used as organic light-emitting materials. When the compounds are coupled with the scraper coating technique, an organic light-emitting element having a multi-layered structure is obtained without miscibility among the layers in an all-solution state. As such, the film is formed by small molecules. Further, the method of the present invention forms an element or device having a large surface area and lower production cost.

### BRIEF DESCRIPTION OF THE DRAWINGS

The present invention can be more fully understood by reading the following detailed description of the preferred embodiments, with reference made to the accompanying drawings, wherein:

FIG. 1 is a sectional view showing the structure of an organic light-emitting element of the present invention;

FIG. 2 is a sectional view showing the structure of another organic light-emitting element of the present invention;

FIG. 3 is a schematic diagram illustrating the step of coating a solution of organic molecules by using a scraper of the present invention;

FIG. 4 is a comparative curve diagram of organic light-emitting elements obtained according to an all-solution process of the present invention and a conventional vapor deposition process;

FIG. 5 is another comparative curve diagram of organic light-emitting elements obtained according to an all-solution process of the present invention and a conventional vapor deposition process; and

FIG. 6 is a spectrogram of organic light-emitting elements obtained according to an all-solution process of the present invention and a conventional vapor deposition process.

### DETAILED DESCRIPTION OF THE PREFERRED EMBODIMENTS

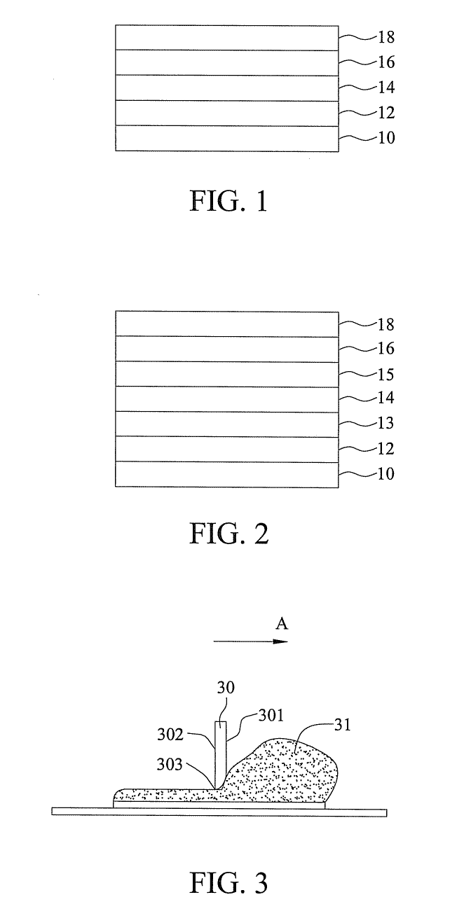

Illustrative embodiments of an organic light-emitting material, an organic light-emitting element using the same and a method of forming the same of the present invention are described as follows with reference to FIGS. 1 to 6. It should be understood that the drawings are simplified schematic diagrams only showing the components relevant to the present invention, and the layout of components could be more complicated in practical implementation.

The present invention provides a compound of formula (I):

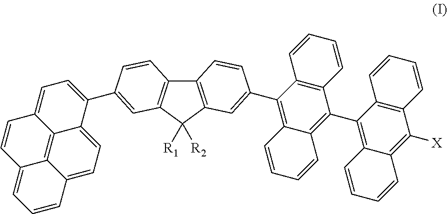

wherein R₁ and R₂ are each a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group.

For example, the linear or branched alkyl group includes the followings, but is not limited to: a methyl group, an ethyl group, an n-propyl group, an isopropyl group, an n-butyl group, an isobutyl group, a sec-butyl group, a t-butyl group, an n-pentyl group, an iso-pentyl group, a neo-pentyl group, a t-pentyl group and a hexyl group.

In addition to a hydrogen atom, X can be groups or compounds having an elongated conjugative structure such as a phenyl group or a biphenyl group.

In a preferred embodiment, the compound of formula (I) of the present invention is a compound of the following formulae (a), (b), (c), (d) or (e):

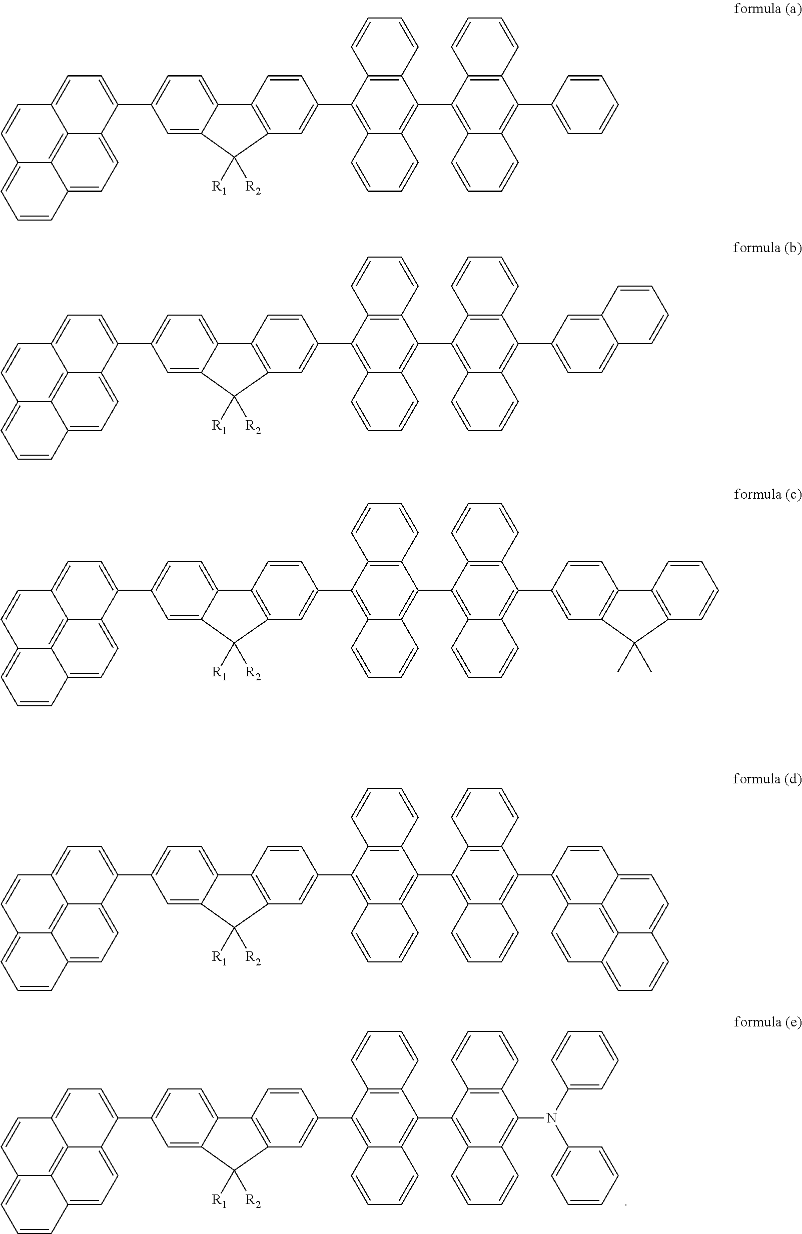

The compound of formula (I) of the present invention can be used as a host material in a light-emitting layer of an organic light-emitting element.

The present invention further provides a compound of formula (II):

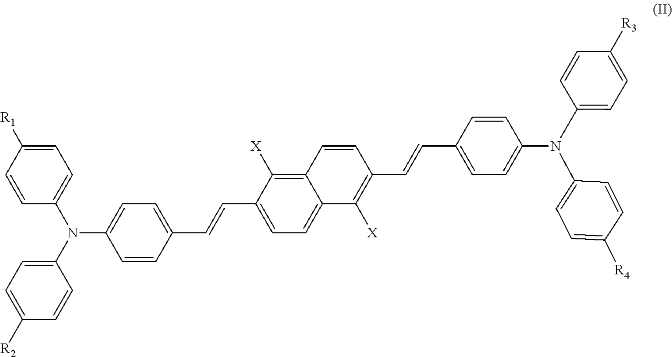

wherein R₁, R₂, R₃, and R₄ each have a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group.

For example, the linear or branched alkyl group includes the followings, but is not limited to: a methyl group, an ethyl group, an n-propyl group, an isopropyl group, an n-butyl group, an isobutyl group, a sec-butyl group, a t-butyl group, an n-pentyl group, an iso-pentyl group, a neo-pentyl group, a t-pentyl group and a hexyl group.

In addition to a hydrogen atom, X can be a phenyl group or other aromatic rings. For example, the compound of formula (II) can be a compound of formulae (f) or (g):

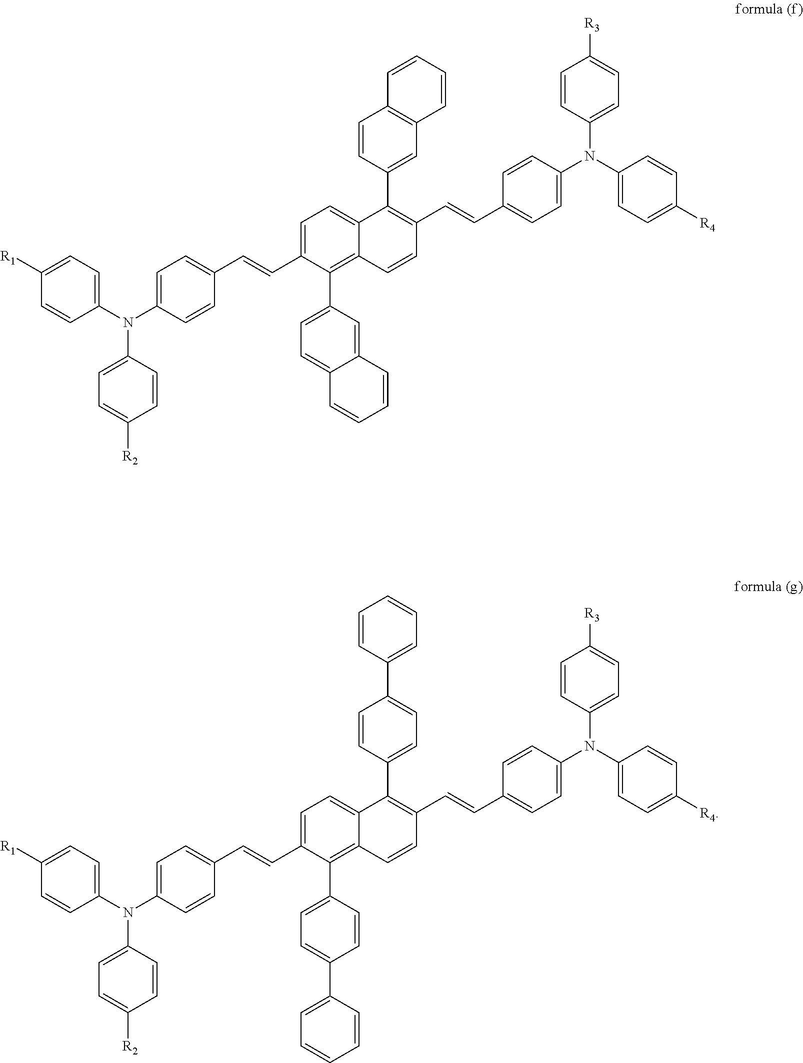

The compound of formula (II) can be used in a dopant material in a light-emitting layer of an organic light-emitting element, to form a composition with other organic light-emitting materials and then to form a light-emitting layer. More specifically, the compound of formula (II) is used as a guest material in a light-emitting layer, and forms a composition with the compound of formula (II) to give a blue light organic material having high luminous efficiency.

In a preferred embodiment of the present invention, a light-emitting layer comprises a compound of formula (I) and a compound of formula (II), wherein the compound of formula (II) has a weight ranging from 0.5 to 5 wt %, based on the weight of the compound of formula (I), to increase the luminous efficiency of a photoelectronic element.

The present invention further provides an organic light-emitting element. As shown in FIG. 1, the organic light-emitting element of the present invention comprises a first electrode 10, a first carrier transport layer 12, a light-emitting layer 14, a second carrier transport layer 16 and a second electrode layer 18. The organic light-emitting element of the present invention has a sandwich structure, wherein the light-emitting layer 14 having a compound of formula (I) and a compound of formula (II) of the present invention is disposed between the first electrode 10 and the second electrode 18; the first carrier transport layer 12 is formed between the light-emitting layer 14 and the first electrode 10; and the second carrier transport layer 16 is formed between the light-emitting layer 14 and the second electrode 18.

As shown in FIG. 2, another organic light-emitting device of the present invention further comprises the existent first electrode 10, the first carrier transport layer 12, the light-emitting layer 14, a first carrier blocking layer 13 disposed between the light-emitting layer 14 and the first carrier transport layer 12, the second carrier transport layer 16 and the second electrode 18. Moreover, the organic light-emitting element can further comprises a second carrier blocking layer 15 disposed between the light-emitting layer 14 and the second carrier transport layer 16.

Specifically, the first electrode is a cathode, and the second electrode is an anode. The anode comprises a lithium fluoride layer disposed on the inner side of the organic light-emitting element and an aluminum layer disposed on the outer side of the organic light-emitting element. In this embodiment, the first carrier transport layer is a hole transport layer, and the second carrier transport layer is an electron transport layer. The first carrier blocking layer is an electron blocking layer, and the second carrier blocking layer is a hole blocking layer.

In order to obtain the organic light-emitting element of the present invention, the present invention provides a method for fabricating an organic light-emitting element. Referring to FIG. 1, the method of the present invention comprises the following steps of: providing a substrate (not shown), and forming a first electrode 10 on a surface of the substrate and forming a first carrier transport layer 12 on the first electrode 10; injecting a solution of organic molecules on the first carrier transport layer 12; and coating the solution of organic molecules on the substrate to form a wet coating layer; heating the wet coating layer to remove the solvent to form a light-emitting layer 14; forming a second carrier transport layer 16 on the light-emitting layer 14; and forming a second electrode 18 on the second carrier transport layer 16, wherein the solution of organic molecules comprises a compound of formula (I) and a compound of formula (II).

In order to obtain the organic light-emitting element shown in FIG. 2, the present invention further comprises the step of forming a first carrier blocking layer 13 prior to injecting the solution of organic molecules, such that the first carrier blocking layer 13 is disposed between the light-emitting layer 14 and the first carrier transport layer 12. Similarly, the method further comprises the step of forming a second carrier blocking layer 15 prior to forming a second carrier transport layer 16, such that the second carrier blocking layer 15 is disposed between the light-emitting layer 14 and the second carrier transport layer 16.

According to the fabrication process of the element, the first electrode is usually a cathode made of a transparent conductive material such as indium tin oxide (ITO), and the second electrode is usually an anode. In a preferred embodiment, the anode comprises a lithium fluoride layer disposed on the inner side of the organic light-emitting element and an aluminum layer disposed on the outer side of the organic light-emitting element, in addition to being a commonly used cesium fluoride anode. Moreover, as shown in an aspect shown in FIG. 2, the first carrier transport layer is usually a hole transport layer, and the second carrier transport layer is usually an electron transport layer.

Although the present invention does not discuss the fabrication of the other layers (e.g., the first carrier transport layer and the second carrier transport layer) in details except for the light-emitting layer, the fabrication of the other layers can all involve in a step similar to the steps of forming a light-emitting layer (i.e., coating a solution to form coating layer) during fabrication. That is, the steps of dissolving a carrier transport material in an organic solvent, coating a solution containing the carrier transport material onto a surface to be coated, uniformly coating the solution on the surface to form a wet coating layer, and then heating the wet coating layer to remove the solvent to obtain a desirable coating layer.

On the other hand, a gap between the scraper and the substrate is greater than or equal to 30 μm, so as to form a coating layer having a more uniform thickness. Generally, the thickness at different locations in the entire coating layer can be controlled to within 10 nm. It is similar in the embodiments, wherein the gap is 50 μm, 90 μm or even 120 μm.

Preferably, the bit of the scraper is a linear structure shown in FIG. 3. As compared with a conventional planar scraper (i.e., the contact with a solution occurs on a plane), a linear scraper or a knife-shaped scraper can be used to reduce the wave patterns on a coating surface, so as to produce a more uniform coating effect. In a preferable embodiment, a scraper 30 coats in a direction indicated by arrow A. The scraper 30 has a first surface 301 for coating a solution 31 of organic molecules and a second surface 302 opposing to the first surface 301. The converged site on the first and second surfaces 301, 302 is a linear or knife-shaped bit 303. In a preferred embodiment, the site on the second surface 302 that is where coated solution is found is a flat surface. As compared with a rod-shaped scraper having an arc contact surface, the flat surface can indeed eliminate the wave patterns. The elimination of the patterns occurs as a result of an included angle between the flat second surface and the coated solution (i.e., wet coating layer) being greater than that between the arc contact surface and the coated solution, and/or the second surface is approximately perpendicular to, or even forms an obtuse angle with, the substrate or the surface of the coated solution. In the view from the device, the site on the second surface that is close to the substrate is a flat surface, and the included angle between the second surface and the substrate is approximately a straight angle.

In conclusion, when an organic light-emitting element having a multi-layered structure is fabricated according to the method of the present invention, the steps of injecting a solution of organic molecules, coating using a scraper and heating are repeated, so as to form an organic light-emitting device having a multi-layered structure. Of course, the repetition of the above steps can result in the formation of an organic light-emitting element having a desirable number of layers, and form a uniformly coated multi-layered structure by an all-solution process. Thus, the process of the present invention is applicable to the fabrication of a photoelectronic element having a large surface area.

Generally, a hot plate, an infrared heater and a hot-air heating device can be used to perform heating. Further, the temperature for heating a wet coating layer can be set at a range from 40° C. to 800° C. Preferably, the temperature can be set at a range from 40° C. to 200° C.

The following examples further illustrate the present invention, but they are only used for exemplification without intending to limit the scope of the present invention.

### SYNTHESIS EXAMPLE 1

### SYNTHESIS OF A REPRESENTATIVE COMPOUND OF FORMULA (I)

### Step 1

100 ml of toluene and 50 ml of ethanol were added to a 250 ml three-necked flask. Deaeration was performed for 30 minutes by adding nitrogen gas. In the presence of nitrogen gas, 4.9 g of pyrene-1-boronic acid (20 mmol), 12.1 g of 7-dibromo-di-n-octylfluorene (22 mmol), 0.2 g of tetrakis triphenyl palladium (Pd(PPh₃)₄) and 50 ml of 2 M sodium carbonate (Na₂CO₃) solution were added thereto, and stirred overnight while the temperature reached 60° C. to obtain a reaction solution. The reaction solution was filtered, and then extracted with water and toluene. The obtained organic layer was dewatered, evaporated under a reduced pressure, and then purified by using a silica gel column to give 7.8 g of a product, 2-bromo-7-pyrenyl-9,9-di-n-octylfluorene (yield: 58%), which had a structure of the following formula.

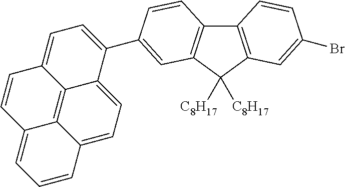

### Step 2

A 100 ml three-necked flask was dewatered. In the presence of nitrogen, 50 ml of dewatered tetrahydrofuran was added thereto. Then, 6.7 g of 2-bromo-7-pyrenyl-9,9-di-n-octylfluorene (10 mmol) was added, and stirred until complete dissolution was reached. The temperature was cooled to −70° C. An amount of 6.3 ml of 1.6 M n-butyl lithium (10 mmol) was added slowly and dropwisely, and stirred for 1 hour. Then, 1.6 g of trimethyl borate was further added dropwisely at −70□, and stirred overnight while the temperature naturally rewarmed to obtain a reaction solution. The reaction solution was acidified by using 50 ml of 2 M hydrochloric acid. The obtained aqueous layer was removed. The obtained organic layer was concentrated to give 5.8 g of a product, 7-pyrenyl-9,9-n-octylfluorene-2-boronic acid (yield: 91%), which had a structure of the following formula. The following step was performed directly without purifying the product.

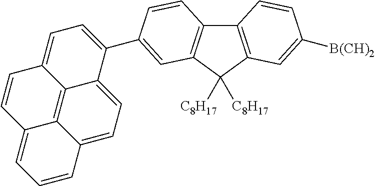

### Step 3

100 ml of toluene and 50 ml of ethanol were added to a 250 ml three-necked flask. Deaeration was performed for 30 minutes by adding nitrogen gas. In the presence of nitrogen gas, 5.7 g of 7-pyrenyl-9,9-di-n-octylfluorene-2-boronic acid (9 mmol), 3.9 g of 10-bromo-9,9-bianthrane (9 mmol), 0.2 g of Pd(PPh₃)₄ and 23 ml of 2 M Na₂CO₃ solution were added, and stirred overnight while the temperature reached to 60° C. to obtain a reaction solution. The reaction solution was filtered, and the obtained solid was washed by dichloromethane. The obtained organic layers were combined, dewatered, evaporated under a reduced pressure, and then purified by using a silica gel column to give 3.9 g of a product, 1-(7-(9,9′-bianthracenyl-10-yl)-9,9-dioctyl-9H-fluorene-2-yl)pyrene (yield: 46.3%), which had a structure of the following formula.

Analytical Data:

FAB MS: m/z=943, 500 Hz NMR in CDCl3: 0.87(t, 6H), 1.28˜1.32(m, 24H), 1.85(t, 4H), 7.35[7.50(m, 12H), 7.56(d, 1H), 7.60˜7.63(m, 2H), 7.75(d, 1H), 7.78˜7.81(d, 2H), 7.99˜8.27(m, 13H), 8.52 (s, 1H)

UV/PL measured in tetrahydrofuran: 257 nm/422 nm ;

DSC decomposition temperature: 340□ (0.5% weight loss)

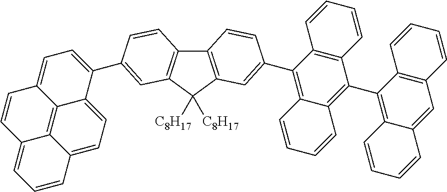

### SYNTHESIS EXAMPLE 2

### SYNTHESIS OF A REPRESENTATIVE COMPOUND OF FORMULA (II)

### Step 1

A 500 ml round-bottomed flask was dewatered, and then 20 ml of dimethyl formamide (DMF) was added thereto. In an ice bath, 15.3 g of phosphorus oxychloride (POCl₃) (0.1 mmol) was added dropwisely, and stirred for 10 minutes at a temperature ranging from 5 to 10° C. after the addition was completed. An amount of 38 g of N-phenyl-N,N-di(4-n-hexylphenyl)aniline (91 mmol) was dissolved in 200 ml of DMF to obtain a mixture. The mixture was added slowly and dropwisely into the flask. After the addition was completed, heating was performed at a temperature ranging from 60 to 70° C., and a reaction took place overnight to obtain a reaction solution. The reaction solution was slowly poured into 1 L of water, neutralized to a reach neutral pH by using 20 wt % of a sodium hydroxide solution, and extracted with ethyl acetate. The obtained organic layer was concentrated under a reduced pressure, and then purified by using a silica gel column to give 29.6 g of a product (yield: 73%) having a structure of the following formula.

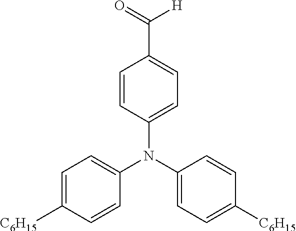

### Step 2

3.1 g of 2,6-di(bromomethyl)naphthalene (10 mmol) and 30 ml of triethyl phosphate were added to a 100 ml three-necked flask. A reaction took place for 2 hours after the temperature was elevated under reflux. Then, the solvent was obtained by steaming under low vacuum, and subsequently removed. The residue was dissolved in 60 ml of dewatered tetrahydrofuran, and together poured into a baked 500-ml three-necked flask. An amount of 200 ml of the dewatered tetrahydrofuran and 9.8 g of the product (22 mmol) obtained in step 1 were added thereto, and thoroughly mixed. 4.5 g of potassium t-butoxide was further added, and reacted overnight as the temperature reached 60° C. to obtain a reaction solution. The reaction solution was extracted with water and dichloromethane. The obtained organic layer was dewatered, extracted under a reduced pressure, and then purified by using a silica gel column to give 4.2 g of a product, 4,4′-(1E,1′E)-2,2′-(naphthalene-2,6-diyl)bis(ethylene-1,2-diyl)bis(N,N-bis(4-hexylphenyl))aniline (yield: 41.5%), which had a structure of the following formula.

Analytical Data:

FAB MS: m/z=1011; 500 Hz NMR in CDCl3: 0.86(m, 12H), 1.28˜1.37(m, 24H), 1.62 (m, 8H), 2.58 (t,8H), 6.54˜6.68 (m, 12H), 6.88˜6.93(d, 4H), 7.09(m, 8H), 7.67˜7.73(m, 6H), 7.85(d, 2H), 7.91(s, 2H) UV/PL measured in tetrahydrofuran: 414 nm/475 nm;

DSC decomposition temperature: 330□ (0.5% weight loss)

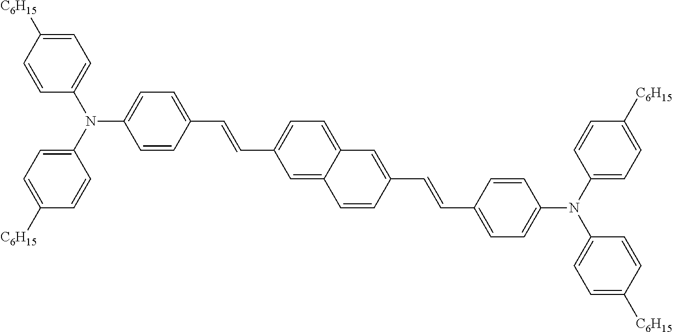

### SYNTHESIS EXAMPLE 3

### SYNTHESIS OF A REPRESENTATIVE COMPOUND Of FORMULA (a)

### Step 1

100 ml of toluene and 50 ml of ethanol were added to a 250 ml three-necked flask. Deaeration was performed for 30 minutes by adding nitrogen gas. In the presence of nitrogen gas, 4.9 g of pyrene-l-boronic acid (20 mmol), 12.1 g of 7-dibromo-di-n-octylfluorene (22 mmol), 0.2 g of tetrakis triphenyl palladium (Pd(PPh₃)₄) and 50 ml of 2M sodium carbonate (Na₂CO₃) solution were added thereto, and stirred overnight while the temperature reached 60° C. to obtain a reaction solution. The reaction solution was filtered, and then extracted with water and toluene. The obtained organic layer was dewatered, evaporated under a reduced pressure, and then purified by using a silica gel column to give 7.8 g of a product, 2-bromo-7-pyrenyl-9,9-n-octylfluorene (yield: 58%), which had the structure of the following formula.

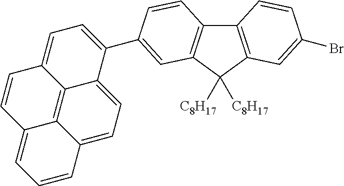

### Step 2

A 100 ml three-necked flask was dewatered. In the presence of nitrogen, 50 ml of dewatered tetrahydrofuran was added thereto. Then, 6.7 g of 2-bromo-7-pyrenyl-9,9-di-n-octylfluorene (10 mmol) was added, and stirred until complete dissolution was reached. The temperature was cooled to −70° C. 6.3 ml of 1.6 M n-butyl lithium (10 mmol) was added slowly and dropwisely, and stirred for 1 hour. Then, 1.6 g of trimethyl borate was further added dropwisely at −70° C., and stirred overnight while the temperature naturally rewarmed to obtain a reaction solution. The reaction solution was acidified by using 50 ml of 2 M hydrochloric acid. The obtained aqueous layer was removed. The obtained organic layer was concentrated, to give 5.8 g of a product, 7-pyrenyl-9,9-n-octylfluorene-2-boronic acid (yield: 91%), which had a structure of the following formula. The following step was performed directly without purifying the product.

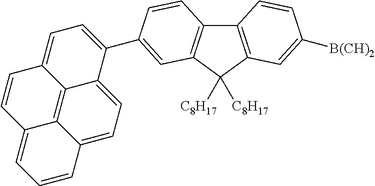

### Step 3

100 ml of toluene and 50 ml of ethanol were added to a 250 ml three-necked flask. Deaeration was performed for 30 minutes by adding nitrogen gas. In the presence of nitrogen gas, 6.35 g of 7-pyrenyl-9,9-di-n-octylfluorene-2-boronic acid (10 mmol), 5.1 g of 10-bromo-10′-phenyl-9,9-bianthrane (10 mmol), 0.2 g of Pd(PPh₃)₄ and 20 ml of 2M Na₂CO₃ solution were added thereto, and stirred overnight while the temperature reached 60° C. to obtain a reaction solution. The reaction solution was filtered, and the obtained solid was washed by dichloromethane. The obtained organic layers were combined, dewatered, evaporated under a reduced pressure, and then purified by using a silica gel column to give 3.6 g of a product (yield: 35.3%) having a structure of the formula (a).

Analytical Data:

FAB MS: m/z=1019, 500 Hz NMR in CDCl₃: 0.87(t, 6H), 1.28˜1.32(m, 24H), 1.85(t, 4H), 7.35˜7.57(m, 18H), 7.60˜7.63(m, 2H), 7.75(d, 1H), 7.78˜7.81(d, 2H), 7.99˜8.27(m, 13H)

UV/PL in tetrahydrofuran: 262 nm/430 nm;

DSC decomposition temperature: 360□ (0.5% weight loss)

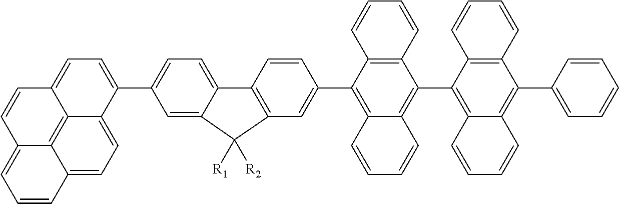

### SYNTHESIS EXAMPLE 4

### SYNTHESIS OF A REPRESENTATIVE COMPOUND OF FORMULA (e)

### Step 1

100 ml of toluene and 50 ml of ethanol were added to a 250 ml three-necked flask. Deaeration was performed for 30 minutes by adding nitrogen gas. In the presence of nitrogen gas, 4.9 g of pyrene-l-boronic acid (20 mmol), 12.1 g of 7-dibromo-di-n-octylfluorene (22 mmol), 0.2 g of tetrakis triphenyl palladium (Pd(PPh₃)₄) and 50 ml of 2 M sodium carbonate (Na₂CO₃) solution were added thereto, and stirred overnight while the temperature reached 60° C. to obtain a reaction solution. The reaction solution was filtered, and then extracted with water and toluene. The obtained organic layer was dewatered, evaporated under a reduced pressure, and then purified by using a silica gel column to give 7.8 g of a product, 2-bromo-7-pyrenyl-9,9-n-octylfluorene (yield: 58%), which had the structure of the following formula.

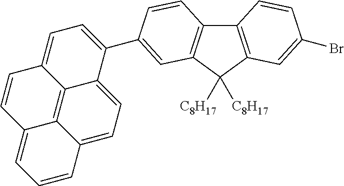

### Step 2

A 100 ml three-necked flask was dewatered. In the presence of nitrogen, 50 ml of dewatered tetrahydrofuran was added thereto. Then, 6.7 g of 2-bromo-7-pyrenyl-9,9-di-n-octylfluorene (10 mmol) was added, and stirred until complete dissolution was reached. The temperature was cooled to −70° C. 6.3 ml of 1.6 M n-butyl lithium (10 mmol) was added slowly and dropwisely, and stirred for 1 hour. Then, 1.6 g of trimethyl borate was further added dropwisely at −70° C., and stirred overnight while the temperature naturally rewarmed to obtain a reaction solution. The reaction solution was acidified by using 50 ml of 2 M hydrochloric acid. The aqueous layer was removed. The obtained organic layer was concentrated, to give 5.8 g of a product, 7-pyrenyl-9,9-n-octylfluorene-2-boronic acid (yield: 91%), which had a structure of the following formula. The following step was performed directly without purifying the product.

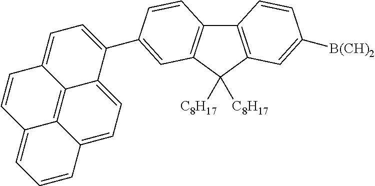

### Step 3

100 ml of toluene and 50 ml of ethanol were added to a 250 ml three-necked flask. Deaeration was performed for 30 minutes by adding nitrogen gas. In the presence of nitrogen gas, 7.0 g of 7-pyrenyl-9,9-di-n-octylfluorene-boronic acid (11 mmol), 6.6 g of 10-bromo-10′-N,N-diphenylamino-9,9-bianthrane (11 mmol), 0.22 g of Pd(PPh₃)₄ and 20 ml of 2 M Na₂CO₃ solution were added thereto, and stirred overnight while the temperature reached 60° C. to obtain a reaction solution. The reaction solution was filtered, and the obtained solid was washed by dichloromethane. The obtained organic layers were combined, dewatered, evaporated under a reduced pressure, and then purified by using a silica gel column to give 4.1 g of a product (yield: 33.6%) having a structure of the formula (e).

Analytical Data:

FAB MS: m/z=1111, 500 Hz NMR in CDCl₃: 0.87(t, 6H), 1.28˜1.32(m, 24H), 1.85(t, 4H), 6.72 (d, 4H), 6.88(m, 2H), 7.15(m, 4H), 7.35˜7.50(m, 12H), 7.56(d, 1H), 7.60˜7.63(m, 2H), 7.75(d, 1H), 7.78˜7.81(d, 2H), 7.99˜8.27(m, 13H),

UV/PL in tetrahydrofuran: 256 nm/435 nm;

DSC decomposition temperature: 360□ (0.5% weight loss)

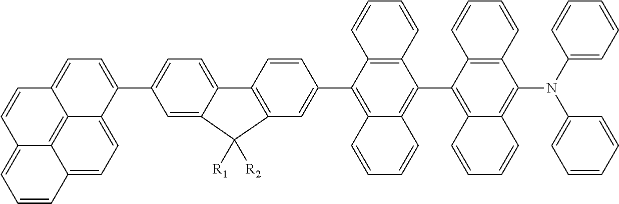

The following examples provide organic light-emitting elements fabricated by an all-solution process of the present invention and a vapor deposition process.

### EXAMPLE 1

### [Fabrication of an Organic Light-Emitting Element by an All-Solution Process]

An ITO-coated glass substrate was provided, and the electrode (cathode) of the substrate was cleaned by using acetone and ultrasound oscillation. The substrate was further cleaned by UV/ozone. Poly(2,4-ethylenedioxythiophene): poly-(styrenesulfonate) (PEDOT: PSS) was spin-coated on the substrate to formula a hole transport layer. Then, 1 wt % of N,N′-bis(naphthalene-1-yl)-N,N′-bis(phenyl)-9,9-dimethyl-1-fluorene (DMFL-NPB) chlorobenzene solution was provided on the hole transport layer, and coated with a scraper to form a wet coating layer (wherein a gap between the scraper and the coated surface is 60 μm). The solvent was removed by heating at 120° C. for 10 minutes. An electron blocking layer having a thickness of 30 nm was formed. Then, the scraper was similarly used to form a light-emitting layer having a thickness of 40 nm. The compounds obtained from synthesis examples 1 and 2 were dissolved in methanol at a weight ratio of 100:2.36, wherein the compounds have a total weight of 0.5 wt % based on the weight of methanol. Then, 1,3,5-tris(N-phenylbenzimidazol-2-yl)benzene (TPBi) was coated by using the scraper, to form an electron transport layer. A conventional method was applied to form a lithium fluoride anode and an aluminum anode sequentially.

### COMPARATIVE EXAMPLE 1

### [Fabrication of an Organic Light-Emitting Element by a Vapor Deposition Process]

A hole transport layer, an electron blocking layer, a light-emitting layer, an electron transport layer and an anode in the structure described in example 1 were formed sequentially on an ITO-coated glass substrate by a conventional vapor depositing method.

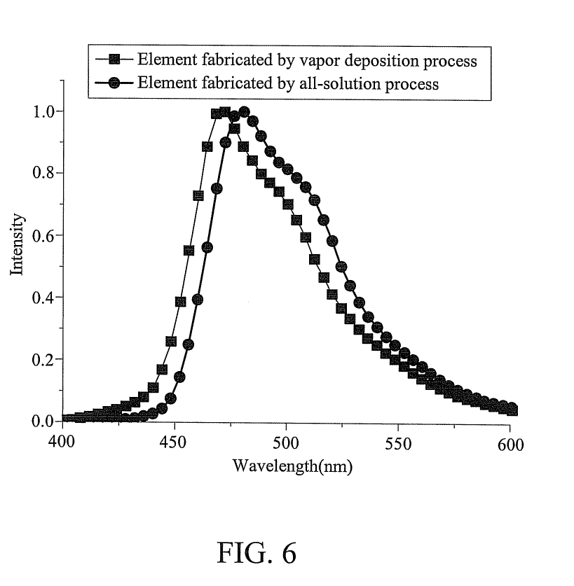

A specific voltage was applied to actuate the organic light-emitting elements fabricated in example 1 and comparative example 1, and the current efficiency and luminance of the elements were measured. A spectrophotometer was used to perform electroluminescent spectroscopic measurements on the elements, and the measured spectra are graphed as shown in FIG. 6.

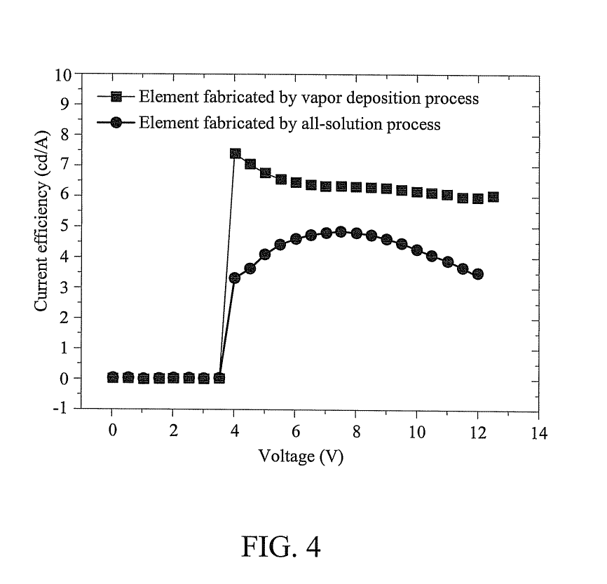

As shown in FIG. 4, at luminance of 1200 cd/cm², the device efficiency of the element fabricated by the all-solution process is 4.8 cd/A, whereas the device efficiency of the element fabricated by the almost vapor deposition process is 6.1 cd/A. Moreover, the fabricating method employing the all-solution process of the present invention has the advantages such as low production cost and rapid processing, such that it is suitable for fabricating an element or device having a large surface area.

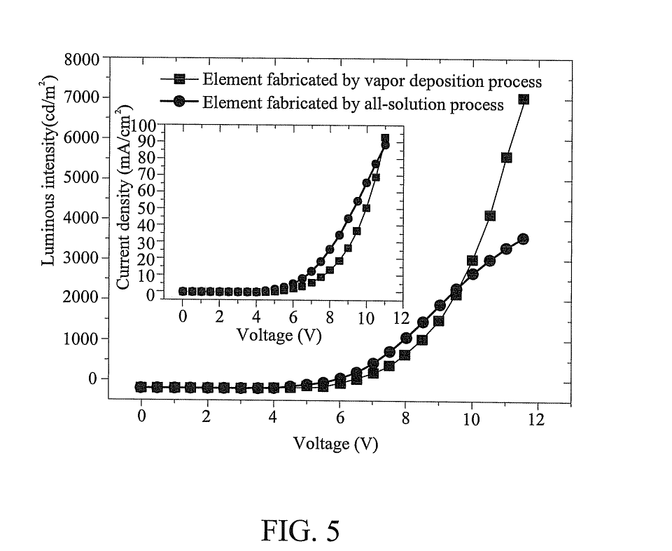

As shown in FIG. 5, the element of the present invention has a current density comparable to that fabricated by the vapor deposition process. As shown in the spectra of the elements in FIG. 6, the element fabricated by the all-solution process has a luminous intensity comparable to that of the element fabricated by the vapor deposition process. In light of the above, it is clear that the compounds of the present invention indeed produce excellent luminous effects, when they are used as organic light-emitting materials for use in a light-emitting layer of a photoelectronic element. Further, there are no obvious red shifts observed in the spectra, indicating that miscibility does not occur among the layers of the element fabricated by the method of the present invention. Accordingly, the present invention uses a scraper coating technique for fabricating an organic light-emitting element to obtain an organic light-emitting element having a multi-layered structure and resolving the miscibility among layers as typically arose from a solution process.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

1. A compound of formula (I): 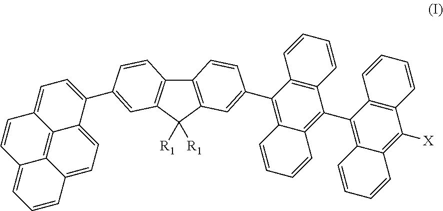
wherein R₁ and R₂ are each a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group.

2. The compound of claim 1, wherein the compound of formula (I) is a compound selected from the group consisting of formulae (a), (b), (c), (d) and (e):

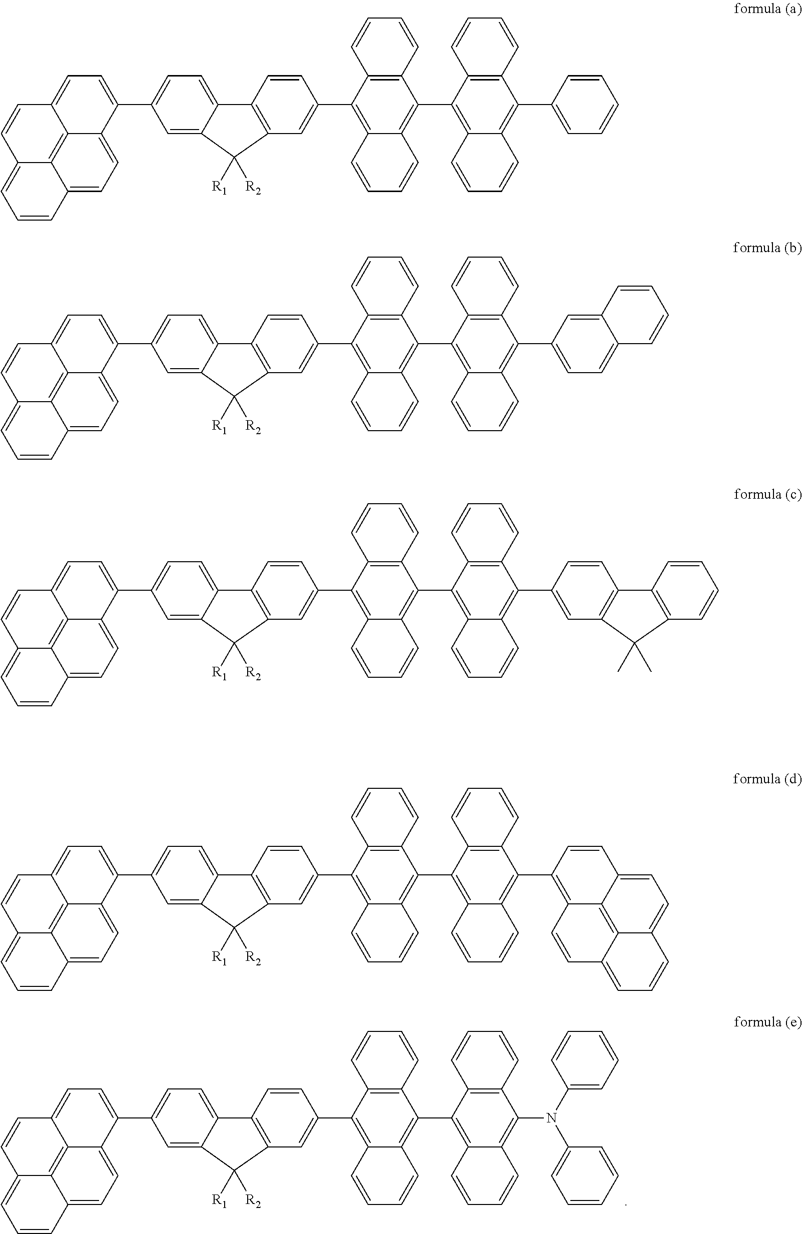

3. The compound of claim 1, which is used in a light-emitting layer of an organic light-emitting element.

4. The compound of claim 3, which is used as a host material of the light-emitting layer.

5. A compound of formula (II): 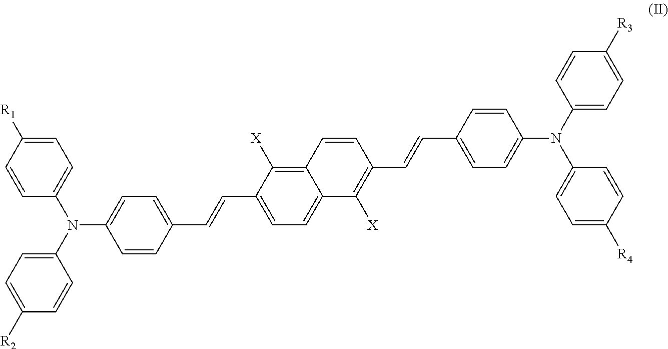
wherein R₁, R₂, R₃, and R₄ each have a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group.

6. The compound of claim 5, wherein X is a phenyl group.

7. The compound of claim 5, wherein the compound of formula (II) is a compound of one of formulae (f) and (g):

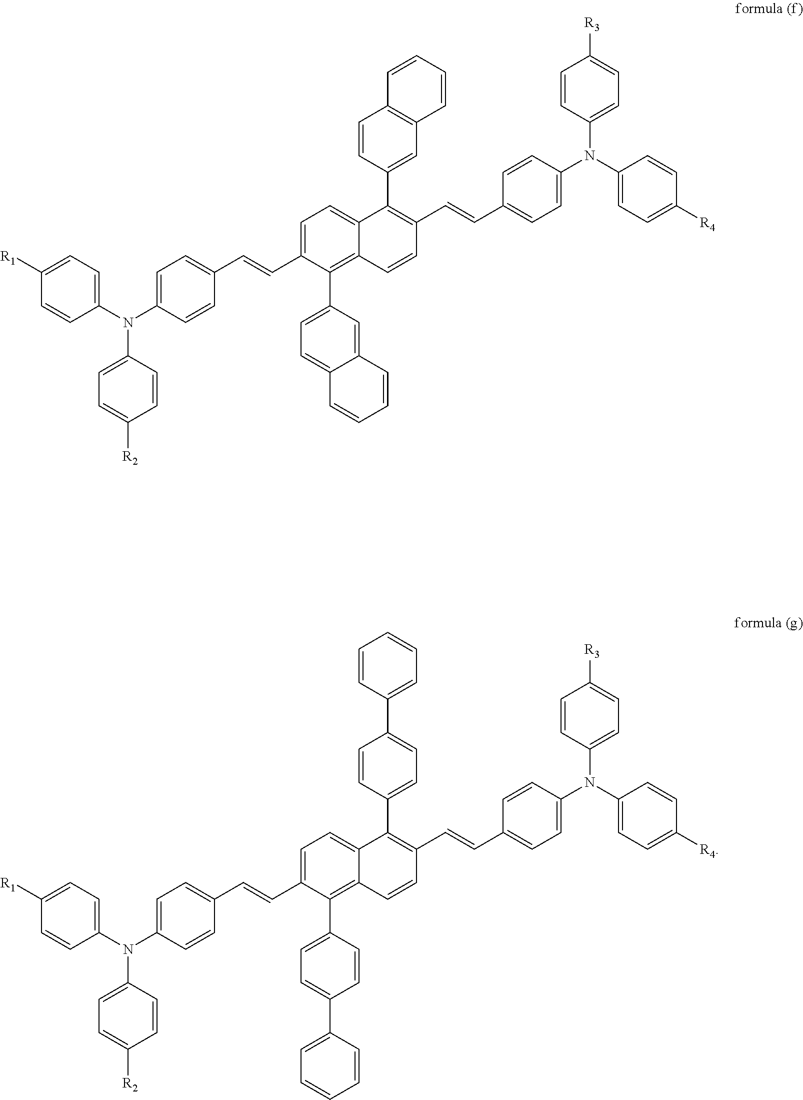

8. The compound of claim 5, which is used in a light-emitting layer of an organic light-emitting element.

9. The compound of claim 8, which is used as a host material of the light-emitting layer.

10. An organic light-emitting element, comprising:
(a) a first electrode;
(b) a second electrode;
(c) a light-emitting layer disposed between the first electrode and the second electrode and comprising:
(i) the compound of the following formula (I) 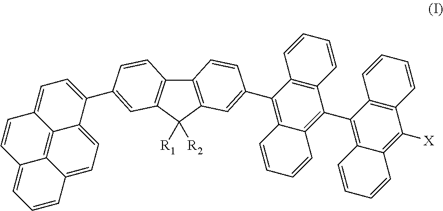
wherein R₁ and R₂ are each a linear alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group; and
(ii) the compound of formula (II) of claim 5l
(d) a first carrier transport layer formed between the light-emitting layer and the first electrode; and
(e) a second carrier transport layer formed between the light-emitting layer and the second electrode.

11. The organic light-emitting element of claim 10, wherein the compound of formula (II) has a weight ranging from 0.5 to 5 wt % based on a weight of the compound of formula (I).

12. The organic light-emitting element of claim 10, further comprising a first carrier blocking layer disposed between the light-emitting layer and the first carrier transport layer.

13. The organic light-emitting element of claim 12, further comprising a second carrier blocking layer disposed between the light-emitting layer and the second carrier transport layer.

14. The organic light-emitting element of claim 10, wherein the first electrode is a cathode, and the second electrode is an anode.

15. The organic light-emitting element of claim 14, wherein the anode further comprises a lithium fluoride layer disposed on an inner side of the organic light-emitting element, and an aluminum layer disposed on an outer side of the organic light-emitting element.

16. The organic light-emitting element of claim 15, wherein the first carrier transport layer is a hole transport layer, and the second carrier transport layer is an electron transport layer.

17. A method for fabricating an organic light-emitting element, comprising the following steps of:
(a) providing a substrate having a first electrode formed on a surface thereof and a first carrier transport layer formed on the first electrode, and providing a solution of organic molecules on the first carrier transport layer, wherein the solution of organic molecules comprises:
(i) the compound of the following formula (I) 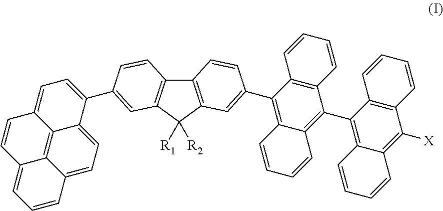
wherein R₁ and R₂ are each a linear or branched alkyl group having 1 to 12 carbon atoms, and X is one selected from the group consisting of a hydrogen atom, a linear or branched alkyl group having 1 to 12 carbon atoms, an aryl group having 6 to 16 carbon atoms, a heterocyclic group containing one of N, O and S, cyano, a substituted amino group and a substituted silyl group, and
(ii) the compound of formula (II) of claim 5;
(b) coating the solution of organic molecules on the substrate by using a scraper, to form a wet coating layer;
(c) heating the wet coating layer to remove a solvent to form a light-emitting layer;
(d) forming a second carrier transport layer on the light-emitting layer; and
(e) forming a second electrode on the second carrier transport layer.

18. The method of claim 17, wherein the compound of formula (II) has a weight ranging from 05 wt % to 5 wt %, based on a weight of the compound of formula (I).

19. The method of claim 17, further comprising the step of forming a first carrier blocking layer prior to providing the solution of organic molecules, such that the first carrier blocking layer is disposed between the light-emitting layer and the first carrier transport layer.

20. The method of claim 17, further comprising the step of forming a second carrier blocking layer prior to forming the second carrier transport layer, such that the second carrier blocking layer is disposed between the light-emitting layer and the second carrier transport layer.

21. The method of claim 17, wherein the first electrode is a cathode, and the second electrode is an anode, and wherein the anode comprises a lithium fluoride layer disposed on an inner side of the organic light-emitting element and an aluminum layer disposed on an outer side of the organic light-emitting element.

22. The method of claim 21, wherein the first carrier transport layer is a hole transport layer, and the second carrier transport layer is an electron transport layer.

23. The method of claim 17, wherein the first carrier transport layer is formed by coating a solution with the scraper.

24. The method of claim 17, wherein the second carrier transport layer is formed by coating a solution with the scraper.
